(19) United States
(12) Patent Application Publication
(10) Pub. No.: US 2008/0090769 A1
(43) Pub. Date: Apr. 17, 2008

(54) TREATMENT OF PSORIASIS THROUGH DOWN-REGULATION OF THE EGF-RECEPTOR WITH TOPICALLY APPLIED EGF

(76) Inventor: Rudi D. Neirinckx, Zollikon (CH)

Correspondence Address: BELL, BOYD, & LLOYD LLP, P.O. BOX 1135, CHICAGO, IL, 60690, US

(21) Appl. No.: 11/954,006

(22) Filed: Dec. 11, 2007

Related U.S. Application Data
(63) Continuation of application No. 11/464,401, filed on Aug. 14, 2006, now abandoned.
Application No. 11/464,401 is a continuation of application No. 11/242,358, filed on Oct. 3, 2005, now Pat. No. 7,306,357.
Application No. 11/242,358 is a division of application No. 09/584,978, filed on Jun. 2, 2000, now Pat. No. 7,015,199.

(51) Int. Cl. A61K 38/18; A61P 17/06

(52) U.S. Cl. 514/12

(57) ABSTRACT

From unrelated but similar fields it is deduced that certain forms of psoriasis can be effectively treated through topical application of EGF-containing formulations. This patent application summarizes the theoretical basis for this finding and requests protection for the idea, while clinical evaluation is in preparation.

### INTRODUCTION

Psoriasis is a chronic skin disorder that affects approximately 4.0 million people in the US., and annual treatment costs in the USA alone are estimated at over $1.5 billion. There are no currently available drugs for this disease that offer satisfactory efficacy and safety. Psoriatic lesions are caused by the hyperproliferation of keratinocytes, but it has been demonstrated that EGF-R signalling is required for the growth of keratinocytes.

It has been demonstrated that the upper epidermal layer in psoriatic tissue contains levels of EGF receptors (EGFR) more similar to the levels found in the mitotically-active basal cell layer of skin. In normal epidermis r-EGF is located primarily in the germinative layer, which contains r-EGF levels 4 times higher than those found in the more-diferentiated cells of the upper epidermal layers. In psoriatic lesions the upper epidermal layers shows r-EGF levels 2× higher than in normal tissue, while the germanitive layer has normal levels. (L. A. Nanney et al; J. Invest. Dermat. Vol 85, p 260-265).

There is only a poor correlation between the levels of r-EGF and the level of cellular proliferation. An example of cells with elevated metabolism but low mitotic activity is the case of the sweat duct epithelium. Similarly, the high level of r-EGF indicates elevated metabolism rather than lack of differentiation in psoriatic lesions.

### PROPOSAL

As the main difference in r-EGF distribution in normal and psoriatic tissue is the abnormal retention of the receptor beyond the first 2-3 cell rows in the stratum basilis in psoriatic tissue, we propose to reduce these concentrations through a down regulation of the receptor using higher than normal levels of EGF at the level of the receptor.

This is similar to the down regulation of FSH and LH excretion through the saturation of pituitary GnRH receptors in response to a constant level of GnRH.

This down regulation is due to the deviation from the normal physiological situation where intermittent surges of GnRH release LH and FSH, without causing saturation of the receptors. It is also similar to the effect of high levels of estradiol on estrogen-dependent tumour lines: In-vitro, the proliferation of these cells can be halted by high, non-physiological concentrations of the hormone.

It has been reported that high levels of EGF have inhibited the growth of EGF-dependent cancer cell lines in-vitro. The biological activity of epidermal growth factor (EGF) is mediated through the intrinsic tyrosine kinase activity of the EGF receptor (EGFR). In numerous cell types, binding of EGF to the EGFR stimulates the tyrosine kinase activity of the receptor eventually leading to cell proliferation. In tumor-derived cell lines, which overexpress the EGFR, however, growth inhibition is often seen in response to EGF. The mechanism for growth inhibition is unclear. A constant pressure of EGF may engender a similar down regulation of the EGF receptors and result in a more-normal metabolic activity and a reversion of psoriatic tissue to normal.

### CLINICAL EVALUATION

Two patients, suffering from psoriasis, were treated with a topical cream containing sulfadiazine and 5 μg of EGF/gram of cream. The treatment was carried out by applying 2 grams of cream over each psoriatic lesion and was carried out twice a day for a week.

### RESULTS AND CONCLUSION

After a week's treatment the psoriatic lesions showed—subjectively—a marked improvement, comparable to the result obtained after treatment with corticosteroids.

It is therefore felt that a larger clinical trial is warranted.

1: A topical formulation comprising epidermal growth factor (EGF) and a dermatologically acceptable excipient or carrier.

2: The formulation of claim 11, wherein said EGF is in an amount to down regulate the EGF receptor.

3: The formulation of claim 11, wherein said EGF is present at 0.01 to 50 μg/g.

4: The formulation of claim 13, wherein said EGF is present at 0.5-20 μg/g.

5: The formulation of claim 11, further comprising an anti-inflammatory.
